(19) United States
(12) Patent Application Publication
(10) Pub. No.: US 2020/0297518 A1
(43) Pub. Date: Sep. 24, 2020

(54) METHOD AND DEVICE FOR DETERMINING A SUFFICIENT STENT REMOVAL FORCE

(71) Applicant: BIOTRONIK AG, BUELACH (CH)

(72) Inventors: HEINZ ANDEREGG, BREMGARTEN (CH); ILONA RUEGER, BERINGEN (CH); BEAT LEHNER, NIEDERROHRDORF (CH); DENNIS VONDENDRIESCH, NEFTENBACH (CH)

(21) Appl. No.: 16/771,322

(22) PCT Filed: Nov. 29, 2018
(86) PCT No.: PCT/EP2018/082975

§ 371 Date: Jun. 10, 2020

(30) Foreign Application Priority Data
Dec. 14, 2017 (EP) 17207299.3

(51) Int. Cl. A61F 2/958; G01N 3/08

(52) U.S. Cl. CPC A61F 2/958; A61F 2240/008; A61F 2002/9583; G01N 3/08

(57) ABSTRACT

A method for determining a sufficient stent removal force includes providing a balloon fastened to a shaft with a stent securely clamped to the balloon. In order to move or remove the balloon from the stent, a stent removal force has to act on the balloon in the axial direction. The stent, which surrounds the balloon, is clamped between first and second holding jaws with a predefined contact pressure perpendicularly to the axial direction. A predefined, desired stent removal force is then exerted on the balloon via the shaft in the axial direction, at which the balloon should not be removed from the stent. If the balloon moves axially relative to the stent, the force is considered to be too low and the shaft-balloon-stent assembly is discarded as a reject. If the stent position remains constant, the stent removal force is considered to be sufficient.

The present invention relates to a method and a device for determining a sufficient stent removal force or stent retention force, also referred to hereinafter as an implant removal force. The invention will also be described hereinafter on the basis of the example of a balloon-expandable stent and an associated balloon catheter, but is not limited in principle to this application and is suitable for determining the removal force of an implant in general.

Stents or vascular supports are used in the field of blood vessel stenosis in order to hold open the constricted vessel in question or to expand the flow cross-section. The stent in question is for this purpose crimped for example onto a balloon, which is inflatable via a shaft connected to the balloon, such that the stent arranged in the constricted vessel region is expandable to an extended diameter so as to hold open the constricted point of the vessel.

An important variable in respect of a stent of this kind is what is known as the stent retention force (SRF), which will also be referred to herein as the stent removal force. This stent removal force refers to a force that has to be exerted on the stent in order to move it axially on the balloon or to remove it from the balloon. In order to determine this force, the stent can be held for example, wherein a tensile force is exerted on the balloon in the axial direction. This tensile force can be exerted on the balloon for example via the shaft extended in the axial direction.

Current methods for determining a sufficient stent removal force determine this force for example as follows. In accordance with a first variant the stent-balloon combination is fixed in the centre of a U-shaped holder by means of two adhesive strips.

The holder is pulled by means of a certain force, whilst the shaft (balloon) is held at the other end. If the holder is pulled by the desired stent removal force, the stent must remain on the balloon or must not move axially thereon. Otherwise, the test is not deemed to be passed.

Alternatively, in accordance with a further test method, the balloon-stent combination is for this purpose left in the crimping device and the shaft is pulled on by a predefined force.

There is also the possibility to act on the stent struts using a hook-shaped tool in order to remove the stent from the balloon.

A disadvantage of the aforementioned methods is in particular the fact that the fixing of the stent-balloon combination in the aforementioned holder has to be performed manually and is therefore costly. Furthermore, said adhesive strips can not only act on the stent, but also on the balloon arranged therebeneath, which increases the susceptibility to errors of the method accordingly.

Proceeding from this basis, the problem addressed by the present invention is that of providing an improved method and an improved device for determining a sufficient stent removal force.

This problem is solved by a device having the features of claim 8 and by a method having the features of claim 1. Advantageous embodiments of the respective aspects of the invention are stated in the corresponding dependent claims and will be described hereinafter.

In accordance with claim 1, a method for determining a sufficient removal force is disclosed, comprising the following steps:
providing an implant (100) securely clamped to a catheter part, wherein the catheter part and implant extend along an axial direction (z), such that a removal force (F_{A}) on the catheter part in the axial direction (z) has to be exceeded in order to move the catheter part relative to the implant in the axial direction (z),
clamping the implant securely clamped on the catheter part between a first and a second holding jaw (10, 20) with a predefined contact pressure (F_{B}) perpendicularly to the axial direction (z),
exerting a predefined, desired removal force (F_{A}) on the catheter part in the axial direction (z), at which force the catheter part should not move relative to the implant in the axial direction (z), wherein, if the catheter part moves relative to the implant in the axial direction (z), the removal force (F_{A}) of the implant is considered to be too low, and wherein if the position of the implant relative to the catheter part is constant, the removal force (F_{A}) of the implant (100) is considered to be sufficient.

The invention will be described hereinafter on the basis of the example of a method for determining the stent removal force for a stent which is securely clamped on a balloon which is fastened to a catheter shaft. Accordingly, everything explained hereinafter for a balloon fastened to a catheter shaft and a stent securely clamped thereon is to be understood synonymously for a catheter part and an implant securely clamped thereon. However, the invention is suitable in particular for determining the stent removal force for a stent securely clamped on a balloon.

Here, movement of the balloon relative to the stent when said predefined, desired stent removal force is exerted is also considered to be a complete removal of the balloon from the stent.

In accordance with one embodiment of the method according to the invention, it is also provided that the holding jaws merely contact or hold the stent. In this regard it is provided in particular that the two holding jaws contact or hold merely an outer side of the stent, which runs around the balloon in a peripheral direction running perpendicular to the axial direction. The holding jaws therefore in particular do not directly hold the balloon or the shaft extending from the balloon.

In accordance with one embodiment of the invention it is also provided that the two holding jaws are each pressed with a surface against the stent with the contact pressure, wherein said surface is formed from one of the following materials: silicone, polyurethane or other elastomers.

In accordance with a further embodiment of the method according to the invention it is provided that the first holding jaw is pressed against the stent by means of a pretensioned spring in order to exert the contact pressure, such that the first holding jaw presses the stent against the second holding jaw and the balloon surrounded by the stent is thus clamped between the two holding jaws by the contact pressure.

It is furthermore provided in the method according to one embodiment that the axial direction runs vertically, wherein in particular the stent removal force exerted on the balloon points downwardly in the vertical direction.

A further aspect of the present invention relates to the provision of a measurement standard device which defines a measurement standard. This device can be used to exert a precisely predefinable tensile force on the balloon.

This measurement standard device can be inserted between a free end of the shaft and a fastening device, wherein a tensile force is exerted on the measurement standard device via the fastening device in the axial direction. This tensile force is also introduced into the free end of the shaft and thus into the balloon via the measurement standard device.

The measurement standard device in accordance with one embodiment comprises a first portion and a second portion connected thereto, wherein the two portions detach from one another when they are pulled apart from one another with a predefined force. A predefined tensile force can thus be exerted on the shaft/balloon in the axial direction, since the two portions separate from one another when this force is reached.

In accordance with one embodiment the two portions can each be formed by a magnet, wherein the two magnets are attracted to one another by a predefinable force. The force necessary to separate the two portions/magnets can be adjusted for example by a corresponding choice of the magnets. The measurement standard device can also comprise more than two magnets.

With use of the measurement standard device, it is possible to exert a tensile force on the measurement standard device in the axial direction in any way (i.e. manually or by machine). The tensile force is exerted or increased here such that the measurement standard device opens, i.e. the two portions of the measurement standard device are detached from one another. Should the force necessary to open the measurement standard device correspond to the stent removal force that is to be tested or that is desired, it can be determined whether the stent has a sufficient stent removal force. Should the stent move axially or be removed from the balloon before the measurement standard device opens (i.e. the two portions of the measurement standard device are detached from one another), the stent removal force is not sufficient, and the balloon-stent combination is discarded as a reject.

A further aspect of the present invention relates to a device for determining a sufficient stent removal force. This device can be used in particular to carry out the method according to the invention.

The device according to the invention comprises at least:
a clamping device, comprising a first and a second holding jaw, wherein the two holding jaws are opposite one another and are configured to hold a stent surrounding a balloon with a predefinable contact pressure, and
an actuator for exerting a predefinable tensile force on a shaft fastened to the balloon, wherein the actuator comprises a fastening device for fastening the shaft relative to the actuator, such that the tensile force can be exerted on the shaft and thus the balloon via the fastening device.

In accordance with one embodiment of the device according to the invention, it is provided that the first holding jaw comprises a first material region and the second holding jaw comprises a second material region, wherein the two material regions each comprise a surface for contacting and holding the stent, wherein the two surfaces run parallel to one another and perpendicularly to the contact pressure.

The two material regions can be formed for example as a hexahedral pad, although other forms are also conceivable.

It is furthermore provided in accordance with a preferred embodiment of the invention that the two material regions are each formed from one of the following materials: a silicone (i.e. a material from the group of poly(organo)siloxanes) or PUR.

It is furthermore provided in accordance with one embodiment of the device that the first holding jaw comprises a first carrier and that the second holding jaw comprises a second carrier, wherein the two carriers are opposite one another, and wherein the first material region is fastened to the first carrier and the second material region is fastened to the second carrier.

In accordance with a further embodiment of the invention it is provided that the first carrier is fastened to a first arm of the clamping device and that the second carrier is fastened to a second arm of the clamping device.

Here, it is preferably provided in accordance with one embodiment that the first carrier in order to generate the contact pressure can be pretensioned in the direction of the second carrier by means of a spring.

It is furthermore provided in accordance with one embodiment that the spring for adjustment of the contact pressure is supported on a screw that engages via an external thread in an internal thread of the first arm. The spring is preferably arranged here between the first carrier and the screw. By screwing the screw increasingly into the internal thread, the pretension of the spring or the contact pressure is adjustable. Here, however, alternative possibilities for adjusting the force, such as a pneumatic device, are also possible.

Further features and embodiments of the invention will be explained hereinafter with reference to the drawings, in which:

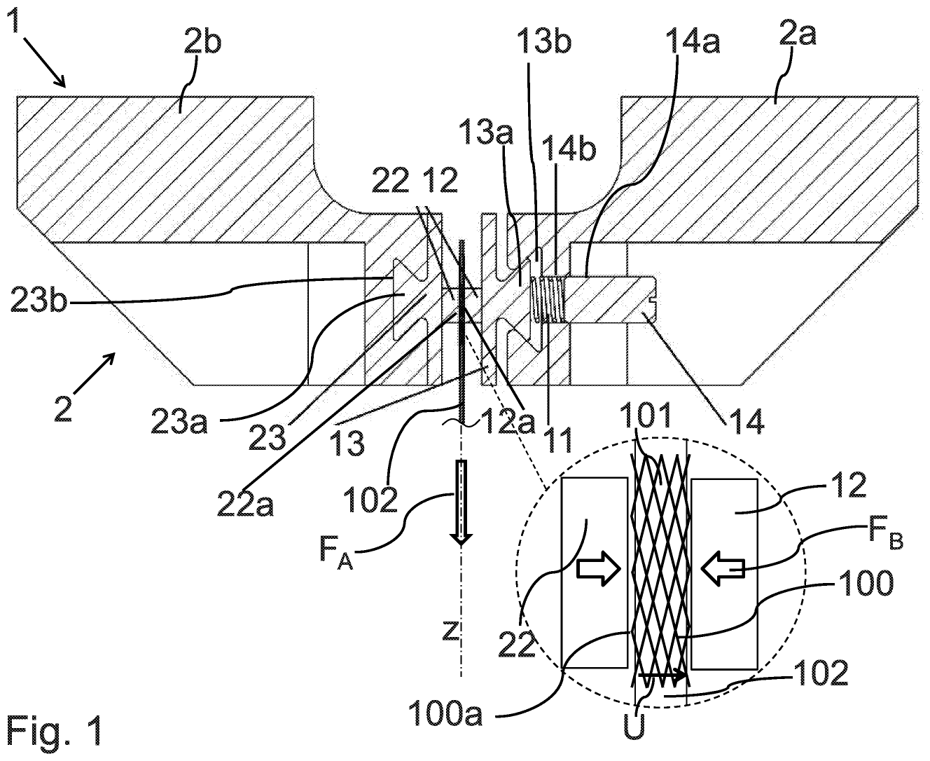

FIG. 1 shows a sectional illustration of a clamping device of a device according to the invention for holding the stent surrounding the balloon; and

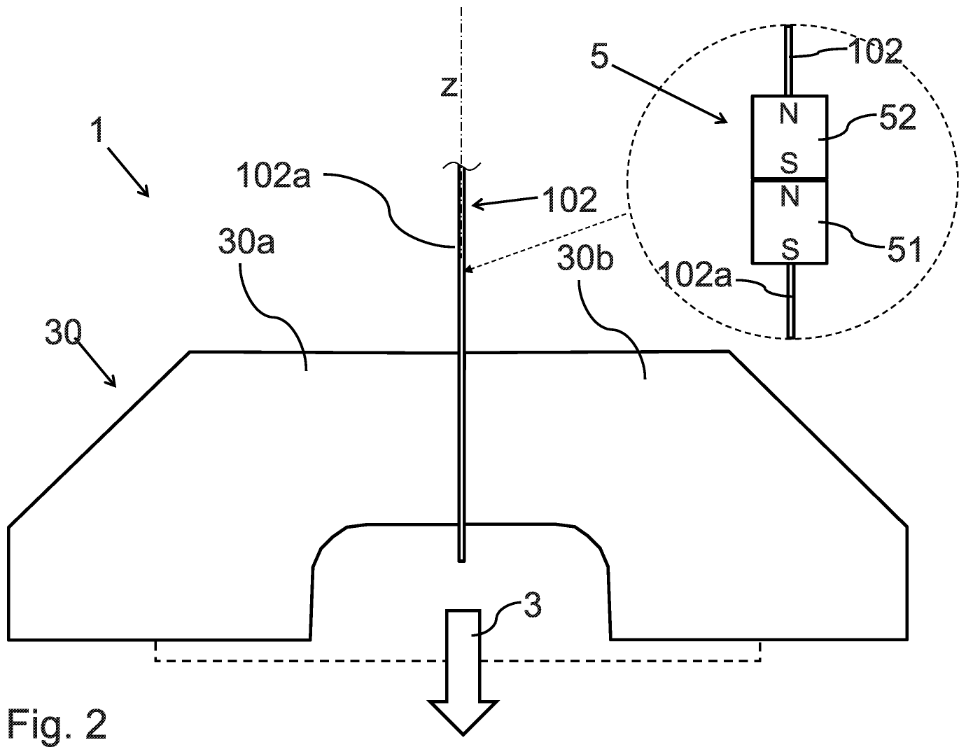

FIG. 2 shows a sectional illustration of a fastening device for introducing a tensile force into the shaft connected to the balloon.

FIG. 1 in conjunction with FIG. 2 shows a device 1 according to the invention for testing the stent removal force of a balloon-stent combination 101, 100. Here, the stent 100 is securely clamped on a balloon 101, such that the stent 100 surrounds the balloon 101 in a peripheral direction U (that is to say transversely to the axial direction z of the stent 100 or of the balloon 101). A shaft 102 extends from the balloon 101 in the axial direction z and can be used to inflate the balloon 101. FIG. 1 in the present case shows an upper part of the device 1, specifically a clamping device 2 for the stent 100. A lower part of the device 1, which is configured to exert a tensile force F_{A} on the balloon 101, which force points downwardly in the axial or vertical direction z, is shown in FIG. 2.

In order to remove the stent 100 from the balloon 101 or conversely the balloon 101 from the stent 100, or in order to move the two components 100, 101 axially relative to one another, what is known as the stent removal force F_{A} has to be applied in the axial direction z, which force must be great enough to ensure the functionality of the balloon catheter (stent-balloon combination 100, 101).

Whether a sufficiently great stent removal force F_{A} is present can be tested in accordance with the invention as follows: Firstly, the stent 100 surrounding the balloon 101 is clamped between a first and a second holding jaw 10, 20 with a predefined contact pressure F_{B} (for example between 1 and 10 N) perpendicularly to the axial direction z. Here, it is preferably provided that the two holding jaws 10, 20 in each case by merely a surface 12a, 22a contact or hold merely an outer side 100a of the stent 100, which surrounds the balloon 101 in the peripheral direction U running perpendicularly to the axial direction z and faces away from the balloon 101. The surfaces 12a, 22a are preferably formed from silicone or PUR or a comparable material.

A free end 102a of the shaft 102 is clamped in accordance with FIG. 2 preferably in a fastening device 30 of an actuator 3. A predefined, desired stent removal force F_{A} (for example between 1 and 15 N, in particular between 10 and 15 N) is then applied to the balloon 101 via the shaft 102 in the axial direction z via the fastening device 30 by means of the actuator 3. If the balloon 101 then moves relative to the stent 100 in the axial direction z, the stent removal force F_{A} of the stent 100 is considered to be too low. Otherwise, the test is deemed to be passed.

According to FIG. 1, it is preferably provided in respect of the clamping device 2 that the first holding jaw 10 comprises a first material region 12 and the second holding jaw 20 comprises a second material region 22, wherein the two material regions 12, 22 are preferably formed as pads and said surfaces 12a, 22a are designed to hold the stent 100. The two surfaces 12a, 22a run parallel to one another and perpendicularly to the contact pressure F_{B}. Said material regions/pads 12, 22 are preferably manufactured from a silicone or from PUR, as already mentioned above.

In order to hold the two material regions 12, 22, the two holding jaws 10, 20 preferably each comprise a carrier 13, 23, wherein the two carriers 13, 23 are opposite one another, and wherein the first material region 12 is fastened to the first carrier 13 and the second material region 22 is fastened to the second carrier 23. Here, the first carrier 13 is fastened to a first arm 2a of the clamping device, wherein in particular the first carrier 13 engages by means of a protrusion 13a in a recess 13b in the first arm 2a. It is also provided that the first carrier 13 in order to generate the contact pressure F_{B} can be pretensioned by means of a spring 11 in the direction of the second carrier 23, wherein the spring 11 in order to adjust the contact pressure F_{B} is supported on a screw 14 which engages by means of an external thread 14a in an internal thread 14b of the first arm 2a. The spring 11 is arranged here between the first carrier 13 and the screw 14. Furthermore, the second carrier 23 can also be fastened to the second arm 2b of the clamping device 2 in that the second carrier 23 engages by means of a protrusion 23a in a recess 23b in the second arm 2b.

According to FIG. 2, the fastening device 30 already mentioned above can also comprise two clamping jaws 30a, 30b for securely clamping a free end 102a of the shaft 2, such that said tensile force or predefined, desired stent removal force F_{A} can be exerted onto the balloon 101 via the fastening device 30. The tensile force F_{A} can be exerted for example by means of an actuator 3 onto the shaft 102 in the axial direction z via the fastening device 30.

FIG. 2 also shows a measurement standard device 5, which can be used to exert a precisely predefinable tensile force on the balloon 101, in particular for the case in which the tensile force F_{A} is not precisely controllable/measurable, for example in the case of a manual exertion of the tensile force F_{A}.

The measurement standard device 5 in accordance with FIG. 2 can be inserted optionally between a free end 102a of the shaft 102a and the fastening device 30, wherein it is possible to exert a tensile force on the measurement standard device 5 in the axial direction via the fastening device 30. This tensile force F_{A} is also introduced into the free end 102a of the shaft 102 and thus into the balloon 101 via the measurement standard device 5.

The measurement standard device 5 can comprise for example a first portion 51 and a second portion 52 connected thereto, wherein the two portions 51, 52 detach from one another when they are pulled apart from one another at a predefined force. In this way, a predefined tensile force F_{A} can be exerted onto the shaft 102 and balloon 101 in the axial direction z, since the two portions 51, 52 separate from one another when this force F_{A} is reached and precisely delimit the exerted force F_{A}. The two portions 51, 52 can be formed for example in each case by a magnet 51, 52, wherein the two magnets 51, 52 are attracted by a predefinable force F_{A}, which is adjustable for example by the choice of the magnets.

1-31 (canceled)

32. A method for determining the sufficiency of a removal force, the method comprising the following steps:
providing an implant securely clamped to a catheter part, wherein the catheter part and the implant extend along an axial direction, and wherein a force acting on the catheter part in the axial direction must exceed a nominal removal force in order to move the catheter part relative to the implant in the axial direction;
clamping the implant, which is securely clamped on the catheter part, between two holding jaws with a predefined contact pressure acting perpendicularly to the axial direction;
exerting a predefined force on the catheter part in the axial direction, the predefined force being a force that does not exceed the nominal removal force and which should not cause the catheter part to move relative to the implant in the axial direction; and:
if the catheter part moves relative to the implant in the axial direction in reaction to the exertion of the predefined force, concluding that the removal force of the implant is too low; and
if the catheter part does not move relative to the implant in reaction to the exertion of the predefined force, concluding that the removal force of the implant is sufficient.

33. The method according to claim 32, wherein the catheter part consists of a shaft and a balloon fastened to the shaft, the implant is a stent, and the removal force is a stent removal force.

34. The method according to claim 33, wherein the clamping step comprises causing the holding jaws to merely contact the stent.

35. The method according to claim 33, wherein the clamping step comprises merely contacting an outer side of the stent with the holding jaws, the stent running around the balloon in a peripheral direction perpendicular to the axial direction.

36. The method according to claim 32, wherein the clamping step comprises causing the holding jaws to merely contact the implant.

37. The method according to claim 32, wherein the clamping step comprises merely contacting an outer side of the implant with the holding jaws, the implant enclosing the catheter part in a peripheral direction perpendicular to the axial direction

38. The method according to claim 32, which comprises pressing each of the holding jaws with a surface against the implant, wherein the surface is formed from a material selected from the group consisting of silicone and PUR.

39. The method according to claim 32, wherein, in order to exert the contact pressure, pressing a first holding jaw against the implant by way of a pretensioned spring, such that the first holding jaw presses the stent against a second holding jaw and the implant surrounding the catheter part is thus clamped between the two holding jaws by the contact pressure.

40. The method according to claim 32, wherein the axial direction is a vertical direction, and the stent removal force exerted on the catheter part points downwardly in the vertical direction.

41. A device for determining a sufficient removal force, the device comprising:
a clamping device having first and second holding jaws disposed opposite one another and configured to hold an implant surrounding a catheter part with a predefinable contact pressure; and
an actuator for exerting a predefinable tensile force on the catheter part, said actuator including a fastening device for fastening the catheter part relative to said actuator, to enable said actuator to exert the tensile force on the catheter part via the fastening device.

42. The device according to claim 41, wherein the catheter part is a balloon and the implant is a stent surrounding said balloon, and wherein said actuator is configured for exerting the tensile force on a shaft that is fastened to the balloon.

43. The device according to claim 41, wherein said holding jaws include a first holding jaw with a first material region and a second holding jaw with a second material region, each of the first and second material regions comprises a surface for contacting and holding the implant, and the first and second surfaces extend parallel to one another and perpendicularly to the contact pressure.

44. The device according to claim 43, wherein the first and second material regions are formed of a material selected from the group consisting of silicone and PUR.

45. The device according to claim 43, wherein said holding jaws comprise a first holding jaw with a first carrier and a second holding jaw with a second carrier opposite said first carrier, and wherein said first material region is fastened to said first carrier and said second material region is fastened to said second carrier.

46. The device according to claim 45, wherein said first carrier is fastened to a first arm of said clamping device, and said second carrier is fastened to a second arm of said clamping device.

47. The device according to claim 46, which comprises a spring disposed to pretension said first carrier in a direction towards said second carrier in order to generate the contact pressure.

48. The device according to claim 47, wherein said spring for adjustment of the contact pressure is supported on a screw that meshes via an external thread in an internal thread of said first arm.
